(19) United States
(12) Patent Application Publication
(10) Pub. No.: US 2024/0035953 A1
(43) Pub. Date: Feb. 1, 2024

(54) MULTILAYER ANALYSIS OF SINGLE CELLS

(71) Applicant: Teracyte Ltd., Yokneam (IL)

(72) Inventors: Gal LENZ, Binyamina-Givat Ada (IL); Yoav NISSAN-COHEN, Mishmar David (IL)

(73) Assignee: Teracyte Ltd., Yokneam (IL)

(21) Appl. No.: 18/486,428

(22) Filed: Oct. 13, 2023

Related U.S. Application Data
(63) Continuation of application No. PCT/IL2022/050388, filed on Apr. 13, 2022.

(51) Int. Cl. G01N 15/14; G01N 21/64

(52) U.S. Cl. CPC G01N 15/1475; G01N 21/6428; G01N 2021/6439

(57) ABSTRACT

Improved methods for analyzing cells by combining information from the same cell before and after lysis. The methods of the present invention allow examination of the interactions or relationships between cellular physiological parameter that can only be measured in living cells (i.e., metabolic state) and the expression of proteins and peptides of interest. Single cell processing is made possible by determination of wells containing single cells therein and avoiding analysis of well containing no or multiple cells therein. Fluorescent and/or bright-field imaging may be used in certain embodiments.

### CROSS-REFERENCE TO RELATED APPLICATIONS

This application is a continuation of International Application No. PCT/IL2022/050388, filed on Apr. 13, 2022, now pending, which claims the benefit of U.S. Provisional Application No. 63/174,556 filed on Apr. 14, 2021, the contents of which are hereby incorporated by reference.

### TECHNICAL FIELD

The present invention relates to improved methods for analyzing cells by combining information from the same cell before and after lysis.

### BACKGROUND

Many techniques are used to detect and measure physical and chemical characteristics of a population of live cells. Flow cytometry (FCM), for example, is a process where a sample is focused to ideally flow one cell at a time through a laser beam, where the light scattered is characteristic to the cells and their components. Cells are often labeled with fluorescent markers so light is absorbed and then emitted in a band of wavelengths. Flow cytometry is routinely used in basic research, clinical practice and clinical trials.

Various systems are commercially available that provide a platform for single cell protein analysis by western analysis. For example, various systems for single cell Western blot (scWestern) analysis are sold under the tradename ProteinSimple including a representative example sold under the trademark MILO™ which uses a multiwell design having 6,400 individual wells per chip. Typically, the diameter of the wells is adapted to accommodate single cells that are sown on these multiwell chips at a dilution that statistically should provide a single cell per well.

However, until now there are no available methods to correlate information obtained from the live cell and the content of the same content after lysis. There is an unmet need for such methods.

### SUMMARY

There are several characteristics and biological functions that can only be measured in functional live cells (i.e., fluorescent dyes, fluorescent reporters), while measurements of most intra-cellular proteins cannot be performed on functional cells as they require cell destruction.

The present invention provides such tools. The present invention provides methods for performing measurements on live and lysed single cells, thereby obtaining physiological and molecular data of a single cell. The measurements are performed on the same cell. Thus, the methods of the present invention provide, for the first time, the ability to measure content and activity of various components of live single cells, lyse said single cells, perform more measurements, match and analyze the information at a single cell resolution. In fact, the methods provide multi-layer analysis of the same cell, both its extracellular and intracellular state.

The teachings of the present invention are advantageous over previously known methods for single cells measurements, in the ability to perform measurements of both live and lysed single cells using the same sample, and match the information received at a single cell resolution, thus providing important previously unattainable information. Today, in order to receive information on live single cells and lysed single cells, one needs to examine live cells and lysed cells separately, mandating the use of larger number of cells, perform more experiments and/or add more repetitions of each experiment. Nevertheless, these methods do not allow correlate the data obtained from live cells to the data obtained from the same lysed cell. The methods described herein provide information of a plurality of live and lysed single cells (and the matching of said information for each single cell), making the information received more reliable, lessens the amount of cells needed and spares additional experiments/repetitions required, compared to cells that are tested separately for their live state and separately for their lysed state.

The methods disclosed herein should have wide ranging applications in the study of the effects of various treatments on cells, in particular mammalian cells, such as required in the pharmaceutical industry, research laboratories etc. Said methods provide a large-scale analysis of a wide range of cell components expressed in live cells and in same lysed cells.

According to one aspect, the present invention provides a method for multi-layer analysis of a chip data obtained from single cell Western blot (scWestern) comprising:

seeding cells in a cell suspension into the wells of the scWestern chip; for each well of the scWestern chip performing a live cell imaging of the seeded cells, which may comprise fluorescent live cell imaging and/or bright field imaging;
responsive to the live cell imaging, receiving a first signal comprising live cell data; lysing the seeded cells;
electrophoretically separating cell content on a gel and immobilizing cell proteome content;
for each well of the scWestern chip staining the proteome content of the cell;
for each well of the scWestern chip, responsive to the staining of the proteome content of the cell, receiving a second signal comprising proteome data;
for each well of the scWestern chip determining the occupancy of the well;
responsive to the determined occupancy information for each well of the scWestern chip, associating the live cell data and the proteome data for each well occupied by at least one cell; and
responsive to the live cell data and the proteome data, outputting chip data of the scWestern chip.
According to some embodiments, determining the occupancy of the well comprises: staining the DNA content in each well of the scWestern chip;
responsive to the staining of the DNA content in each well of the scWestern chip, receiving a third signal comprising DNA data;
for each well of the scWestern chip, comparing the DNA data of the respective third signal to at least one predetermined threshold; and
for each well of the scWestern chip, determining, responsive to an outcome of the respective comparison, occupancy information of the respective well.

According to some embodiments, when determining the occupancy of the well, the method comprises excluding wells that do not contain exactly one cell.

According to some embodiments, the method comprises the bright field imaging is used for identification of well containing no cells therein, one cell therein, and two or more cells therein. Furthermore, according to some embodiments, an image of the plurality of well superimposes a first marking on wells having no cells therein, a second marking on wells having a single cell therein, and a third marking on cells having two or more cells therein.

According to any one of the above embodiments, the live cell imaging comprises fluorescence measurement of live cells. According to some embodiments, staining the cell proteome content comprises fluorescent staining, and wherein receiving the second signal comprises fluorescence measurement. According to some embodiments, the method comprises analyzing the first signal to obtain the live cell data and/or analyzing the second signal to obtain the proteome data.

According to some embodiments, associating the live cell data and proteome data comprises at least one of cohering, comparing, joining and subtracting the data.

According to some embodiments, the method further comprises generating a dataset comprising the chip data for a subset of the wells of the chip. According to some embodiments, the subset of the wells excludes: wells that are not occupied by at least one cell or wells that are occupied by more than one cell; or both.

According to some embodiments, the chip data is selected from the group consisting of first signal, second signal, third signal, analyzed first signal, analyzed second signal, live cell data, proteome data, association between the live cell data and proteome data, and any combination thereof.

According to any one of the above embodiments, the method further comprises determining the occupancy before performing live cell imaging.

According to some embodiments, the cell suspension comprises up to 800,000 cells/ml.

According to any one of the above embodiments, the proteome cell content comprises cell proteins and peptides.

According to any one of the above embodiments, the DNA staining is effected using a fluorescent labelling.

According to some embodiments, the respective DNA data for each well of the scWestern chip comprises a predetermined function of the fluorescence values from the respective well associated with the DNA staining.

According to some embodiments, the predetermined function comprises a multiplication of:

an area under the curve (AUC) of the outcome of a convolution of a signal to noise ratio (SNR) signal and a predetermined peak shape, the SNR signal being of fluorescent counts in relation to a distance from the center of the respective well; and a width of the outcome of the convolution.

According to some embodiments, wherein the at least one predetermined threshold comprises a predetermined statistical attribute of the DNA data of the wells of the scWestern chip.

According to some embodiments, the at least one predetermined threshold comprises a plurality of predetermined statistical attributes, each predetermined statistical attribute associated with a respective one of a plurality of sets of adjacent wells of the scWestern chip.

According to some embodiments, the predetermined statistical attribute comprises a median value.

According to some embodiments, wherein the at least one predetermined threshold comprises the lowest value of the DNA data of the wells of the scWestern chip.

According to some embodiments, the at least one predetermined threshold comprises the lowest value of the DNA data of each of a plurality of sets of adjacent wells of the scWestern chip.

According to some embodiments, for each of the wells of the scWestern chip, the method further comprises, responsive to an outcome of the comparison to the at least one predetermined threshold, determining whether the respective well is: (i) occupied by one cell, (ii) occupied by more than one cell or (iii) not occupied.

According to some embodiments, the method further comprises, for each of the wells of the scWestern chip determined to be occupied by at least one cell, subtracting from the DNA data of the respective well a respective value associated with the DNA data of at least one well determined to not be occupied by at least one cell.

According to some embodiments, the at least one well not occupied by at least one cell comprises the two of the wells closest to the respective well.

Other objects, features and advantages of the present invention will become clear from the following description and drawings.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows a schematic representation of the proposed multi-layer analysis of cells

FIG. 2A shows the comparison of measurements of citrine from live cell on the chip and on a flow-cytometer.

FIG. 2B shows a scatter plot of citrine quantifications in live cells (Y-Axis) and in by Western-blot performed after lyses (X-Axis) from the same cells (n=1610).

FIG. 3 illustrates a high-level block diagram of a system for analyzing an scWestern chip, in accordance with some embodiments.

FIG. 4 illustrates a high-level flow chart of a method of analyzing an scWestern chip, in accordance with some embodiments.

FIG. 5 illustrates an image of a block well of a 400-well chip after determination and presentation of empty, single and multi-cells wells.

### DETAILED DESCRIPTION

The present invention provides methods for measuring activity and content of live and lysed single cells, and matching the information at a single cell resolution. The methods of the present invention enable the measurements of live single cells signals, including physiological data, that cannot be acquired from lysed cells, such as: cell cycle status, metabolic state, mitochondrial function, metabolite concentration and many other measurements that require an active cell to acquire. This is particularly important when studying metabolic and neurodegenerative diseases (e.g., diabetes, Alzheimer's, Parkinson's). The novel ability of the methods described in the present invention to perform analysis of live single cells, and then match the results with same single cells after undergoing lysis, provides information of multiple parameters on the effect of a treatment on a single cell. Therefore, the present invention fulfills a long-felt need for obtaining information on live and lysed single cells using a single sample and matching the information at a single cell resolution. The methods of the present invention enable the use of various different markers on the same single cells sample, measuring both live and lysed states of the single cells.

The methods of the present invention enable the analysis of living cells thereby providing clinically significant measurements. In addition, the ability to measure protein expression in the Western blot method allows the measurement of a much wider range of purposes and allows the proteins to be characterized even according to parameters of size and modifications (mainly phosphorylation).

The present invention provides a large-scale single cells measurement, whereby each chip contains more than 1500 single cells. This method can also be used in cases where the amount of sample is limited, samples with a small number of cells can be analyzed according to the teachings of the present invention.

According to one aspect, the present invention provides a method for multi-layer analysis of a chip data obtained from single cell Western blot (scWestern) comprising:

seeding cells in a cell suspension into the wells of the scWestern chip;
for each well of the scWestern chip performing a live cell imaging of the seeded cells to obtain a first signal comprising live cell data, which may comprise fluorescent live cell imaging and/or bright field imaging;
lysing the seeded cells;
electrophoretically separating cell content on a gel;
immobilizing cell proteome content; for each well of the scWestern chip staining the cell proteome content;
for each well of the scWestern chip measuring the signal from the staining of the cell proteome content to obtain a second signal comprising proteome data;
associating the live cell data and the proteome data for each well; and
outputting the chip data of the scWestern chip.

According to some embodiments, the method further comprises determining the occupancy of each well of the chip. According to some embodiment, determining the occupancy is effected before live cell imaging, before separating cell content on a gel, before or after staining the cell proteome content, and any combination thereof.

Thus, according to some embodiments, the present invention provides a method for multi-layer analysis of a chip data obtained from single cell Western blot (scWestern) comprising:

seeding cells in a cell suspension into the wells of the scWestern chip;
for each well of the scWestern chip performing a live cell imaging of the seeded cells to obtain a first signal comprising live cell data;
lysing the seeded cells;
electrophoretically separating cell content on a gel and immobilizing cell proteome content;
for each well of the scWestern chip staining the cell proteome content;
for each well of the scWestern chip measuring the signal from the staining of the cell proteome content to obtain a second signal comprising proteome data;
for each well of the scWestern chip determining the occupancy of the well;
responsive to the determined occupancy information for each well of the scWestern chip, associating the live cell data and the proteome data for each well occupied by at least one cell; and
outputting the chip data of the scWestern chip.

As used herein, the term “cell suspension” refers to a type of cell culture in which single cells or small aggregates of cells are allowed to function and multiply in an agitated growth medium, thus forming a suspension. According to some embodiments, the concentration of cells in the cell suspension is up to 1,000,000 cells/ml. According to other embodiments, the concentration of cells in the cell suspension is up to 800,000 cells/ml or up to 600,000 cells/ml. According to one embodiment, the concentration of cells in the cell suspension is up to 500,000 cells/ml. According to another embodiment, the concentration of cells in the cell suspension is up to 400,000 cells/ml. According to yet another embodiment, the concentration of cells in the cell suspension is up to 350,000 cells/ml. According to some embodiments, the concentration of cells in the cell suspension is up to 300,000 cells/ml. According to some embodiments, the concentration of cells in the cell suspension is up to 250,000 of up to 200,000 cells/ml. According to some embodiments, the concentration of cells in the cell suspension is from 10,000 to 1,000,000, from 20,000 to 800,000, from 30,000 to 700,000, from 40,000 to 600,000, from 50,000 to 500,000, from 60,000 to 450,000, from 70,000 to 400,000, from 80,000 to 350,000, from 90,000 to 300,000, from 100,000 to 250,000 from 120,000 to 200,000 cells/ml. According to some embodiments, the concentration of cells in the cell suspension is from 10,000 to 400,000 from 20,000 to 350,000, from 30,000 to 300,000, from 50,000 to 250,000, from 100,000 to 400,000, or from 100,000 to 300,000 cells/ml.

According to some embodiments, the loaded chip comprises from 500 to 4000 cells. According to some embodiments, the loaded chip comprises from 600 to 3500, from 700 to 3000, from 800 to 2500 cells. According to some embodiment, the loaded chip comprises from 900 to 2400, from 1000 to 2000, from 1200 to 1800, or from 1000 to 2500 cells.

According to some embodiments, any type of cell is encompassed. According to some embodiments, the cells are eukaryotic cells. The terms “eukaryotic cells” and “starting eukaryotic cells” according to the present invention include cells isolated (derived) from the above-defined living organisms and cultured in vitro. These cells can be transformed (immortalized) or untransformed (directly derived from living organisms; primary cell culture). The term “eukaryotic cells” also includes mono-cellular eukaryotic cells such as yeasts, etc.

As used herein, the term “bright field imaging” refers to an optical microscopy illumination, also referred to as “bright-field microscopy”, used to illuminate samples with white light and detecting the contrast caused by attenuation of the transmitted light in dense areas of the sample.

As used herein, the term “cell seeding” or the interchangeable term “cell sedimentation” refers to the passive gravity-driven settling of cells in the wells of a scWestern plate. Alternatively, the term may refer to any other force-driven sedimentation.

As used herein, the terms “scWestern plate” and “scWestern chip” are used herein interchangeably and refer to a slide micropatterned with a chip of wells. For examples, the chip may be microscopic slide coated with a thin photoactive polyacrylamide gel (PAG) micropatterned with a chip of wells (i.e., microwells). scWestern plates may be prepared by the surface silanization of a standard glass microscope slide and the epoxy-based negative photoresist (SU-8) mold casting of microwells in a thin PAG layered on the glass microscope slide, to create either a PAGE gel of uniform pore size or a PAGE gel of gradient pore size. Any know scWestern plate may be used in the methods of the present invention, e.g. plate adapted for Milo™ single-cell western (scWestern) platform. Examples of such chips are scWest chip manufactured by ProteinSimple.

The term live cell imaging is well known in art and include many techniques such as ion imaging, fluorescence recovery after photo-bleaching (FRAP), total internal reflection (TIRF), Photoactivation, Multiphoton excitation (MPE), Stimulated emission depletion (STED), Fluorescent lifetime imaging (FLIM), Coherent anti-Stokes Raman Scattering (CARS) and stimulated Raman Scattering (SRS). According to some embodiments, the live cell imaging comprises fluorescent labelling. According to other embodiments, the live cell imaging comprises fluorescence detection. According to some embodiments, the signal obtained from the live cell imaging is a first signal comprising live cell data. According to one embodiment, the fluorescence measured from live cell imaging is a first signal comprising live cell data. Thus, in some embodiments, the method comprises measurement of fluorescence from a live cell imaging of the seeded cells when performing, for each well of the scWestern chip, a live cell imaging of the seeded cells to obtain a first signal comprising live cell data, to obtain a first signal comprising live cell data. According to any one of the above embodiments, the first signal is further analyzed to obtain the live cell data.

According to some embodiments, seeded cells are lysed. Any known methods for lysing cells can be implemented including chemical, enzymatically and physical, e.g., by sonication or homogenization. According to some embodiments, the cells are lysed according to the protocol of a device for single cell Western blotting.

According to some embodiments, the cell content is separated by electrophoresis on a gel or on the chip. Any known methods and techniques for performing electrophoresis may be used for that purpose. In certain embodiments, the gel is a polymeric gel. The polymeric gel may be a gel suitable for gel electrophoresis. The polymeric gel may include, but is not limited to, a polyacrylamide gel (e.g., methacrylamide gel), an agarose gel, and the like. The resolution of the separation medium may depend on various factors, such as, but not limited to, pore size, total polymer content (e.g., total acrylamide content), concentration of cross-linker, applied electric field, assay time, and the like. For instance, the resolution of the separation medium may depend on the pore size of the separation medium. In some cases, the pore size depends on the total polymer content of the separation medium and/or the concentration of cross-linker in the separation medium. In certain instances, the separation medium is configured to resolve analytes with molecular mass differences of 50,000 Da or less, or 25,000 Da or less, or 10,000 Da or less, such as 7,000 Da or less, including 5,000 Da or less, or 2,000 Da or less, or 1,000 Da or less, for example 500 Da or less, or 100 Da or less. In some cases, the separation medium may include a polyacrylamide gel that has a total acrylamide content, T (T=total concentration of acrylamide and bisacrylamide monomer), ranging from 1% to 20%, such as from 3% to 15%, including from 5% to 10%. In some instances, the separation medium has a total acrylamide content of 7.5%. In certain cases, the separation medium has a total acrylamide content of 6%.

According to some embodiments, the content of cells is electrophoretically separated as known in the art. According to other embodiments, the proteome content is immobilized in the gel or chip, as known in the art.

As used herein, the term “PAGE” or “polyacrylamide gel electrophoresis” refers to a technique widely used in biology and biotechnology to separate proteins according to their electrophoretic mobility, and that is included in a step of the scWestern protocol.

As used herein, the term “immunoprobing” refers to incubating the gel with solutions of primary antibodies and then with fluorescently labeled secondary antibodies.

The term “fluorescence” is a well-known term referring to an optical phenomenon in which a molecule absorbs a high-energy photon and re-emits it as a lower-energy (longer-wavelength) photon, with the energy difference between the absorbed and emitted photons ending up as molecular vibrations or heat. The terms “fluorescence value”, “fluorescence count” and “fluorescence signal” may be used interchangeably and refer to the values obtained from measurement the fluorescence intensity. As used herein, the term “sum of fluorescence” refers to the area under the curve (AUC), calculated from a curve obtained by plotting the signal-to-noise ratio of a fluorescence signal along its migration distance.

As used herein, the term “immunoprobing” refers to incubating the gel with solutions of primary antibodies and then with fluorescently labeled secondary antibodies.

The examination of cell-to-cell heterogeneity has contributed greatly to the understanding of cellular processes such as stem cell differentiation and development, immune response, pharmaceutical efficacy, and cancer. When studying the activity of complex cell populations, it is advantageous to use analytical tools offering true single-cell resolution that provide quantitative and highly specific detection of target proteins. Single-cell western blot (scWestern) analysis has emerged as a useful assay for rapid, sensitive, and selective quantitative analysis of protein expression. scWestern is performed on a microdevice that comprises a chip of wells molded in a thin layer of a polyacrylamide gel (PAG). scWestern involves: gravity settling of cells into wells; chemical lysis of cells in each well; PAGE of each single-cell lysate; exposure of the gel to UV light to blot (immobilize) proteins to the gel matrix; and in-gel immunoprobing of immobilized proteins.

The terms “cell proteome content”, “proteome” and “cell proteome” are used herein interchangeably and refers to all peptides and proteins produced in the cell. Any proteins and enzymes are encompassed by this term.

The term “proteome data” refers to data associated with cell proteome content, and specifically to the peptides and proteins tested and analyzed by the methods of the present invention. Protein data encompasses information about the presence or absence of the tested peptides and/or proteins as well as quantitative information about said peptides and/or protein.

According to some embodiments, the peptides and/or proteins are stained. Any known method may be used for that purpose. Non-limiting examples are immuno-staining using antibodies (such as use of primary and secondary antibodies) and ELISA. According to some embodiments, the method comprises use of antibodies labelled with a fluorescent label.

According to some embodiments, the fluorescence from staining the target is detected and measured. According to some embodiments, the method comprises measuring the fluorescence signal obtained from protein, peptide and/or polypeptides staining. According to some embodiments, the second signal comprises the measurement of the fluorescence signal obtained from protein, peptide and/or polypeptides staining. According to some embodiments, the fluorescence signal obtained from protein, peptide and/or polypeptides staining is further processed.

According to some embodiments, for each well of the scWestern chip the method comprises measuring the signal from the staining proteins, peptide or both to obtain a second signal comprising proteome data. According to any one of the above embodiments, the second signal to obtain the proteome content data. According to any one of the above embodiments, the method comprises for each well of the scWestern chip determining the occupancy of the well before or after staining the proteome content of cell.

According to some embodiments, determining the occupancy of the well comprises measuring a DNA data or measuring a protein data in the well.

According to some embodiments, determining the cell occupancy comprises measuring the presence of a house-keeping protein. According to one embodiment, determining the cell occupancy comprises detecting a house-keeping protein. Examples of such proteins are cell skeleton proteins such as beta-tubulin and actin.

According to some embodiments, determining the occupancy of the well comprises measuring a DNA data of cells.

According to some embodiments, the method further comprises, associating the live cell data and the proteome data for each well occupied by at least one cell and outputting the chip data of the scWestern chip. To increase efficacy of the analysis, the associating is performed only to wells comprising at least one cell. Thus, according to some embodiments, the method further comprises, responsive to the determined occupancy information for each well of the scWestern chip. According to some embodiments the association of live cell data and the proteome data for each well is performed for wells comprising exactly one cell. The term “associating the live cell data and the proteome data” refers to cohering, comparing and joining the information obtained from two types of data, as well as subtracting information and any other manipulation that may be performed. Associating the live cell data obtained from a cell with proteome data, i.e., information obtained from the Western blot, for the same cell provides a complete picture of the state of the cell, its reaction to external or internal stimuli etc. The association may be effected by any known method.

According to some embodiments, associating the live cell data with the proteome data comprises generating a dataset where the respective live cell data and proteome data are flagged as belonging to the same cell. According to some embodiments, associating the live cell data with the proteome data comprises, for each cell, comparing a first predetermined attribute of the respective live cell data with a second predetermined attribute of the respective proteome data.

For example, the live cell data can include measurements that quantify certain fluorescent dyes which serve as a proxy to biological functions such as metabolism and proliferation. Comparing the fluorescent measurements associated with the biological functions to protein expression can provide insight into the molecular mechanisms that elucidate those biological functions.

The term “chip data” refers to any data that may be obtained from the Western blot chip, including first signal, second signal, third signal, analyzed first signal, analyzed second signal, analyzed third signal, live cell data, proteome data, association between the live cell data and proteome data, and any combination thereof.

According to some embodiments, the method further comprises generation of a dataset comprising the chip data for a subset of the wells of the chip. According to some embodiments, the dataset comprises the data including first signal, second signal, third signal, analyzed first signal, analyzed second signal, live cell data, proteome data and association between the live cell data and proteome data, and any combination thereof for a subset of the wells of the chip. According to some embodiments, the subset of wells comprises well occupied by at least one cell. According to another embodiments, the subset of wells comprises well occupied by exactly one cell. According to some embodiments, the subset excludes wells that are not occupied by at least one cell.

According to any one of the above embodiments, the method comprises determining occupancy of wells of the chip. As used herein, the term “occupancy” refers to existence of cells in a scWestern plate well following the seeding of a cell suspension into the wells of a scWestern plate. As used herein, the terms “double occupancy”, “double cells occupancy” and “doublet” are used herein interchangeably and refer to presence of two cells in the well of scWestern chip.

According to some embodiments, the method of determining the occupancy of the well comprises:
staining the DNA content in each well of the scWestern chip;
measuring the signal obtained from the DNA staining to obtain a third signalcomprising DNA data;
for each well of the scWestern chip, comparing the DNA data of the respective third signal to at least one predetermined threshold; and
for each well of the scWestern chip, determining, responsive to an outcome of the respective comparison, occupancy information of the respective well.

The method further comprises outputting the occupancy information for each well.

According to some embodiments, the method comprises DNA staining effected using a fluorescent labelling.

According to some embodiments, the method of the present invention comprises staining DNA content in each well of the scWestern chip. Obviously, the staining occurs only in well in which cells were present and lysed. Any known method for staining DNA may be used and a subsequently the corresponding methods for detecting the presence of DNA are utilized. According to some embodiments, DNA staining is effected using fluorescent labelling configured to label DNA. In certain embodiments, the detectable label is a fluorescent label. Fluorescent labels are labeling moieties that are detectable by a fluorescence detector. For example, binding of a fluorescent label to an analyte of interest such as DNA may allow the analyte of interest to be detected by a fluorescence detector. Examples of fluorescent labels include, but are not limited to, fluorescent molecules that emit fluoresce upon contact with a reagent, fluorescent molecules that emit fluoresce when irradiated with electromagnetic radiation (e.g., UV, visible light, x-rays, etc.), and the like. Suitable fluorescent molecules (fluorophores) include, but are not limited to, TOTO®-1 iodide (Quinolinium, 1-1′-[1,3-propanediylbis[(dimethyliminio)-3,1-propanediylbis]]bis[4-[(3-methyl-2(3H)-benzothiazolylidene)methyl]]-, tetraiodide 143413-84), fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1(5,5′,6,6′-tetrachloro-1,1′,3,3′-tetraethylbenzimidazoylcarbocyanine iodide), tetrabromorhodamine 123, rhodamine 6G, TMRM (tetramethylrhodamine methyl ester), TMRE (tetramethylrhodamine ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120),7-amino-4-trifluoromethylcoumarin (Coumarin 151); cyanine dyes; cyanosine; 4′,6-diaminidino-2-phenylindole (DAPI); 5′, 5″-dibromopyrogallol-sulfonaphthalein (Bromopyrogallol Red); 7-diethylamino-3-(4′-isothiocyanatophenyl)-4-methylcoumarin; (dimethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amino-fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; CAL Fluor orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, am nobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP; a GFP from another species such as Renilla reniformis, Renilla mulleri, or Ptilosarcus guernyi; “humanized” recombinant GFP (hrGFP); any of a variety of fluorescent and colored proteins from Anthozoan species; combinations thereof; and the like. In certain embodiments, the method includes introducing a fluid sample into a microfluidic device. Introducing the fluid sample into the microfluidic device may include directing the sample through a separation medium to produce a separated sample. In some cases, the separated sample is produced by gel electrophoresis as the sample traverses the separation medium, as described above. The separated sample may include distinct detectable bands of analytes, where each band includes one or more analytes that have substantially similar properties, such as molecular weight, size, charge (e.g., charge to mass ratio), isoelectric point, etc. depending on the type of gel electrophoresis performed.

According to some embodiments, the DNA is stained by a fluorescent labeling. Any one of the abovementioned labels may be used. According to one embodiment, the fluorescent labeling comprises DAPI. According to another embodiments, the DNA is stained by TOTO-1 iodide. According to some embodiments, the fluorescent label is selected from DAPI, TOTO-1 iodide, Hoechst, 7-ADD, crystal violet, and ethidium bromide.

According to some embodiments, the fluorescence is detected and measured. According to some embodiments, the method comprises measuring the fluorescence signal obtained from DNA staining. According to some embodiments, the fluorescence measuring is effected after immobilizing cell proteome content.

In some embodiments of the method, the label includes a labeled antibody. In some embodiments, the method includes the digital storage of images and pattern processing in a computer system for automated cell processing and analysis.

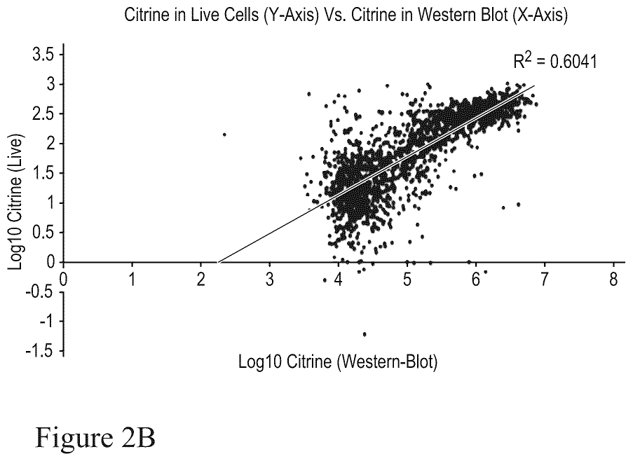

FIG. 3 illustrates a high-level block diagram of a system 10 for analyzing a scWestern chip, in accordance with some embodiments. System 10 comprises: a single cell Western blot platform 20, such as the Milo® platform, including a microarray scanner; a processing system 30; and a memory 40. In one embodiment, processing system 30 and memory 40 are embedded as part of single cell Western blot platform 20. In one embodiment, memory 40 comprises instructions, which when run by one or more processors of processing system 30, cause processing system 30 to perform one or more methods, as described below in relation to stages 400-430.

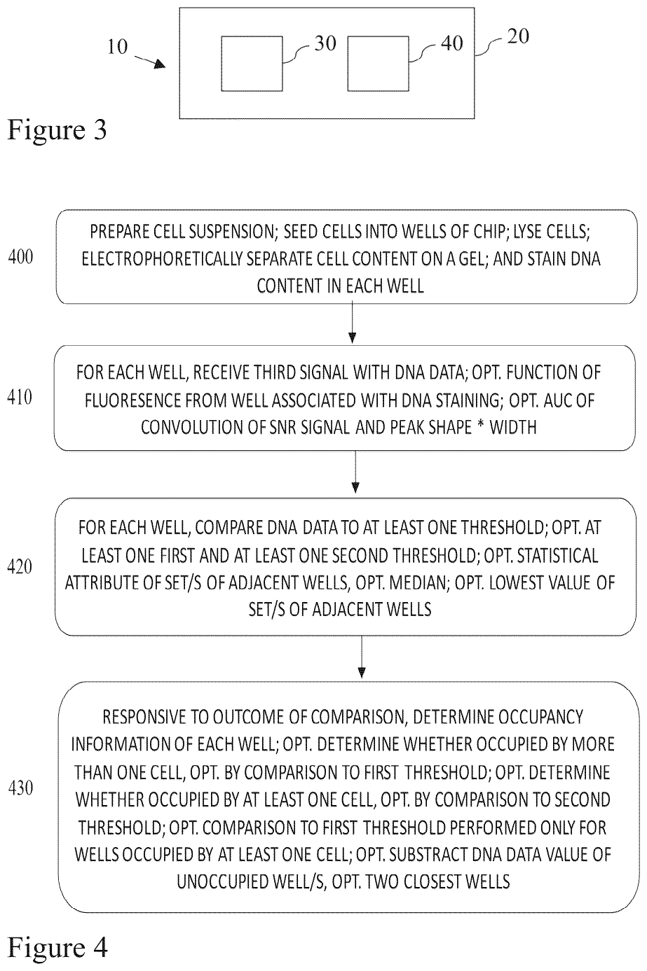

FIG. 4 illustrates a high-level flow chart of a method of analyzing, for example but not by way of limitation, a scWestern chip occupancy using DNA data, in accordance with some embodiments. In stage 400, the DNA content in each well of the chip is then stained with a predetermined DNA dye. In stage 410, for each of a plurality of wells of the chip of stage 400, a respective third signal is received, the respective first signal comprising DNA data regarding the contents of the respective wells. The first signal is received from an output of an imager. The term “DNA data”, as used herein, means data associated with DNA. In one embodiment, the DNA data of each respective first signal comprises a respective value. In another embodiment, the DNA data comprises a predetermined function of the fluorescence within a predetermined wavelength range associated with the predetermined DNA dye. Particularly, the predetermined wavelength range is a wavelength range that the fluorescence of the particular DNA dye is the strongest, as known to those skilled in the art.

In one embodiment, an SNR function is determined, the SNR function being a convolution of: a signal to noise ratio (SNR) signal of the fluorescent counts in relation to a distance from the center of the respective well; and a predetermined canonical peak shape. In one further embodiment, the canonical peak shape is an inverse parabola. In one embodiment, an area under the curve (AUC) of the SNR function is then determined. In one further embodiment, the predetermined function comprises a multiplication of the AUC of the SNR function and the width of the SNR function. In one embodiment, the AUC and the width of the SNR function are calculated between two local minimums of the fluorescence signal. Therefore, according to some embodiments, the DNA data comprises the AUC of the SNR function. In one further embodiment, the SNR function is determined by the Milo® platform, as is the AUC and width thereof.

In stage 420, for each of the plurality of wells of the chip of stage 400, the DNA data of the respective first signal of stage 410 is compared to at least one predetermined threshold. In one embodiment, the at least one predetermined threshold comprises at least one first threshold and at least one second threshold. In one embodiment, the at least one first threshold comprises a predetermined statistical attribute of the DNA data. In one further embodiment, the statistical attribute comprises a mean of the values of the DNA data from one or more sets of wells of the chip. In one further embodiment, where a plurality of sets of wells of the chip are defined, each set of wells is defined as a predetermined number of adjacent wells. In one embodiment, there is a predetermined amount of overlap between adjacent sets of wells. Using a plurality of sets of wells reduces the amount of variability that is naturally present across the chip. In another embodiment, the DNA data values of each well of the chip is determined and each of the plurality of sets of wells is defined such that the variance of the DNA data values in each set is less than a predetermined maximum value. In such an embodiment, the different sets of wells can differ from each other in size and/or shape.

The difference between the value of the DNA data from each well of the chip is the determined mean of the respective set of wells is then determined. In such an embodiment, the first predetermined threshold is determined as a maximum difference from the determined mean. In one further embodiment, the maximum difference is about 1.4-1.8 times the median value, preferably 1.5 times the median value. Although the above embodiment has described in relation to an embodiment where the statistical attribute comprises a mean, this is not meant to be limiting in any way. In another embodiment, the statistical attribute comprises the median, or other statistical function of the values of the DNA data from the one or more sets of wells of the chip.

In one embodiment, the second predetermined threshold is zero. In another embodiment, for each of the one or more sets of wells, the lowest DNA value of the respective set is determined. In such an embodiment, the second predetermined threshold is the lowest DNA value, i.e., the value of the background.

In stage 430, responsive to outcomes of the comparisons of stage 420, occupancy information of each well is determined. In one embodiment, for each of the plurality of wells of the chip of stage 400, responsive to an outcome of the comparison to the at least one first threshold of stage 420, it is determined whether the respective well of the chip is occupied by more than one cell. Particularly, if the respective value of the DNA data is greater than the first predetermined threshold it is due to there being more than one cell in the respective well.

In another embodiment, for each of the plurality of wells of the chip of stage 400, responsive to an outcome of the comparison to the at least one second predetermined threshold of stage 420, it is determined whether the respective well of the chip is occupied by at least one cell. Particularly, if the respective value of the DNA data is greater than the second predetermined threshold, it is due to there being at least one cell in the respective well. Thus, the term “occupancy information”, as used herein, means whether the respective well contains no cells, one cell or more than one cell.

In one embodiment, the comparison of the DNA data to the first predetermined threshold is performed only for cells that have been determined to be occupied by at least one cell.

In another embodiment, prior to the comparison of the DNA data to the first predetermined threshold, for identifying wells being occupied by more than one cell, the background noise is removed. In one embodiment, the background noise is determined for each well by determining the DNA data values of at least one well determined to not be occupied by at least one cell, i.e., wells without cells. In one further embodiment, the DNA data values of the two closest wells not occupied by any cells are determined. A predetermined function of the DNA data values is determined, thereby defining the background noise for the respective well. In one further embodiment, the predetermined function is an average. The background noise value is then subtracted from the DNA data of the respective well, and the subtracted value is compared to the respective first predetermined threshold. In such an embodiment, the first threshold is defined after subtracting the background values for each well.

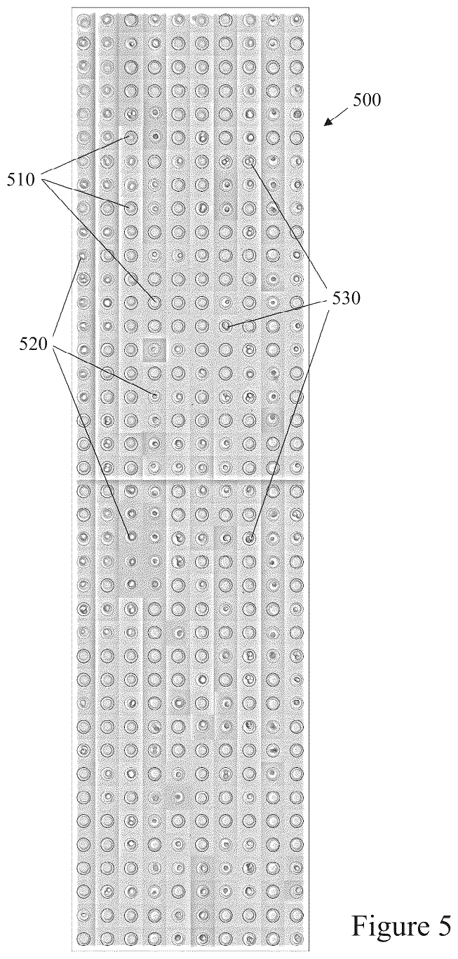

FIG. 5 illustrates an image of a block well of a 400-well chip after determination and presentation of empty, single and multi-cells wells. Using instructions, stored in memory 40, that are executed by the processing system 30, an image of well occupancy of cells may be over imposed with an indication of the number of cells contained within each well. In the case of a Milo chip having 400 well, as shown in the instant illustration, each image of a well is marked by a circle of, for example, a different color, to provide indications of the well's content. For example, but not by way of limitation, a blue circle 510 is drawn around wells that are empty; a green circle 520 drawn around well that contain a single cell, and a red circle 530 drawn around well containing therein two or more cells. Other markings that provide such differentiation between the contents of each well are possible and within the scope of the invention.

The algorithms executed by the processing system 30 may include counting performed by a trained artificial intelligence (AI) model.

According to some embodiments, the method comprises the bright field imaging is used for identification of well containing no cells therein, one cell therein, and two or more cells therein. Furthermore, according to some embodiments, an image of the plurality of well superimposes a first marking on wells having no cells therein, a second marking on wells having a single cell therein, and a third marking on cells having two or more cells therein.

According to some embodiments, the respective DNA data for each well of the scWestern chip comprises a predetermined function of the fluorescence values from the respective well associated with the DNA staining.

According to some embodiments, wherein the predetermined function comprises a multiplication of:
an area under the curve (AUC) of the outcome of a convolution of a signal to noise ratio (SNR) signal and a predetermined peak shape, the SNR signal being of fluorescent counts in relation to a distance from the center of the respective well; and
a width of the outcome of the convolution.

According to one embodiments, the at least one predetermined threshold comprises a predetermined statistical attribute of the DNA data of the wells of the scWestern chip.

According to certain embodiments, the at least one predetermined threshold comprises a plurality of predetermined statistical attributes, each predetermined statistical attribute associated with a respective one of a plurality of sets of adjacent wells of the scWestern chip. According to a further embodiment, the predetermined statistical attribute comprises a median value. According to yet another embodiment, the at least one predetermined threshold comprises the lowest value of the DNA data of the wells of the scWestern chip. According to one embodiment, the at least one predetermined threshold comprises the lowest value of the DNA data of each of a plurality of sets of adjacent wells of the scWestern chip. According to another embodiment, for each of the wells of the scWestern chip, the method further comprises, responsive to an outcome of the comparison to the at least one predetermined threshold, determining whether the respective well is: (i) occupied by one cell, (ii) occupied by more than one cell or (iii) not occupied.

According to yet another embodiment, the method comprises for each of the wells of the scWestern chip determined to be occupied by at least one cell, subtracting from the DNA data of the respective well a respective value associated with the DNA data of at least one well determined to not be occupied by at least one cell. According to yet another embodiment, the at least one well not occupied by at least one cell comprises the two of the wells closest to the respective well.

Thus according to some embodiments, the present invention provides a method for multi-layer analysis of a chip data obtained from single cell Western blot (scWestern) comprising:

seeding cells in a cell suspension into the wells of the scWestern chip;
for each well of the scWestern chip performing a live cell imaging of the seeded cells to obtain a first signal comprising live cell data;
lysing the seeded cells;
electrophoretically separating cell content on a gel and immobilizing cell proteome content;
for each well of the scWestern chip staining the cell proteome content;
for each well of the scWestern chip measuring the signal from the staining of the staining of the cell proteome content to obtain a second signal comprising proteome data;
staining the DNA content in each well of the scWestern chip;
measuring the signal obtained from the DNA staining to obtain a third signal comprising DNA data;
for each well of the scWestern chip, comparing the DNA data of the respective third signal to at least one predetermined threshold;
for each well of the scWestern chip, determining, responsive to an outcome of the respective comparison, occupancy information of the respective well;
responsive to the determined occupancy information for each well of the scWestern chip, associating the live cell data and the proteome data for each well occupied by at least one cell; and
outputting the chip data of the scWestern chip.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

EXAMPLES

### Example 1

Previous work has shown that the promiscuous nature of interactions between the receptors of the Bone morphogenetic proteins (BMP) BMP signaling pathway and a variety of ligands enables a repertoire of computations in response to different environmental conditions. This work has demonstrated that using single-cell measurements of a BMP Response Element (BRE) expression (utilizing a BRE:Citrine reporter protein) can dramatically improve our understanding of the complex data processing capabilities of the cells in response to changing environmental conditions.

Here we suggest a methodology to perform measurements on living individual cells followed by a matched Western-blot quantification of intra-cellular proteins for each of the cells. Allowing us for the first time to correlate between parameters that require living cells with those that require lysed cells.

This would allow us to recreate the measurements of BRE response at single-cell resolution while expending the data set to include other proteins involved in BMP signaling. Improving our understanding of the molecular mechanisms that enable varied computations.

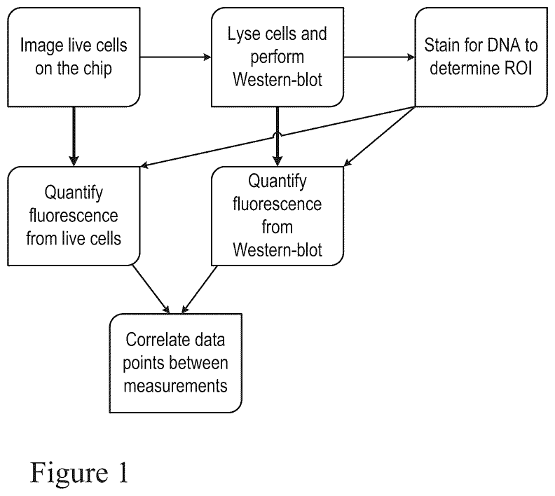

The experiment involved a two-step analysis. First live cells in suspension were loaded onto a single-cell Western-blot chip (Milo™, by ProteinSimple) and a fluorescent image of the chip was generated (InnoScan 710, Innopsys). Following live imaging the cells were lysed in-situ and their lysate was separated and immobilized (crosslinked) onto the chip. Primary antibodies against desired protein targets and fluorescent secondary antibodies were then used to quantify intra-cellular proteins. To make certain that we included only wells occupied with cells during both parts of the analysis, we stained the probed chips with a DNA dye (TOTO1, Invitrogen) and eliminated from the analysis wells with no DNA signal (both live and Western-blot analysis). Schematic representation of the analysis is depicted in FIG. 1.

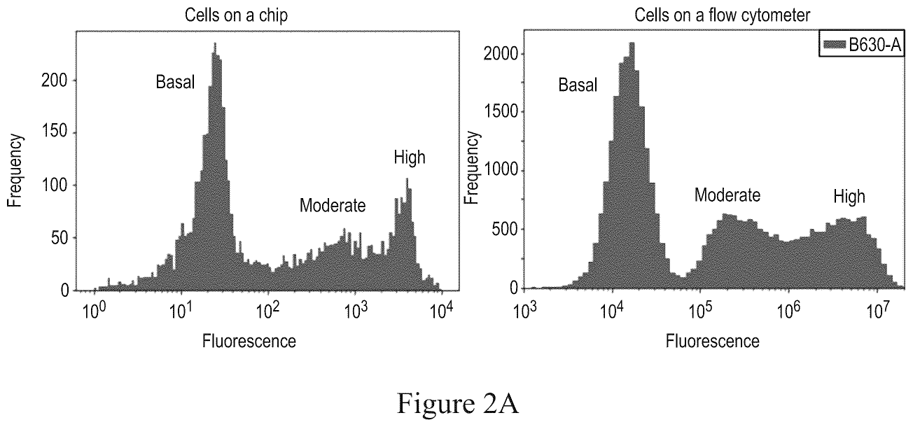

To prove the capacity to accurately quantify fluorescent information from live cells on a chip and the ability to correlate individual data points from each step of the analysis we used D1 cells transformed with a BMP Response Element (BRE):Citrine fusion protein. The cells were stimulated for 48 hours under varied BMP10 concentrations, then mixed into a single sample with a wide range of expected levels of fluorescence. The sample was split in two parts and the measurements of citrine from live cell on the chip were compared to a parallel analysis performed on a flow-cytometer (FIG. 2A). Next, we compared the levels of citrine fluorescence measured in each live cell on the chip with the citrine Western-blot quantifications from the same cell (FIG. 2B).

The tight correlation, particularly in cells with significant citrine expression, of live measurements and Western-blot measurements confirms the capacity to generate two separate measurements for each cell with adequate accuracy. That enables the investigation of interactions or relationships between parameters measured in each step of the analysis, including parameters that can be measured in only one of the measurements.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

What is claimed is:

1. A method for multi-layer analysis of a chip data obtained from single cell Western blot (scWestern) comprising:
seeding cells in a cell suspension into the wells of the scWestern chip;
performing for each well of the scWestern chip, a live cell imaging of the seeded cells;
receiving, responsive to the live cell imaging, a first signal comprising live cell data;
lysing the seeded cells; separating, electrophoretically, cell content on a gel and immobilizing cell proteome content;
staining, for each well of the scWestern chip, the proteome content of the cell;
receiving, for each well of the scWestern chip, responsive to the staining of the proteome content of the cell, a second signal comprising proteome data;
determining for each well of the scWestern chip, the occupancy of the well;
associating, responsive to the determined occupancy information for each well of the scWestern chip, the live cell data and the proteome data for each well occupied by at least one cell; and
outputting, responsive to the live cell data and the proteome data of, chip data of the scWestern chip.

2. The method of claim 1, wherein determining the occupancy of the well comprises:
staining the DNA content in each well of the scWestern chip;
receiving, responsive to the staining of DNA content, a third signal comprising DNA data;
comparing, for each well of the scWestern chip, the DNA data of the respective third signal to at least one predetermined threshold; and
determining, for each well of the scWestern chip, responsive to an outcome of the respective comparison, occupancy information of the respective well.

3. The method of claim 1, wherein the method further comprises:
determining for each well of the scWestern chip, the occupancy of the well, excluding wells that do not contain exactly one cell.

4. The method of claim 1, wherein the live cell imaging comprises fluorescence measurement of live cells. The method of claim 1, wherein staining the cell proteome content comprises fluorescent staining, and wherein receiving the second signal comprises fluorescence measurement.

6. The method of claim 1, wherein the cell proteome content comprises the protein and peptide content of the cell.

7. The method of claim 1, further comprises:
analyzing the first signal to obtain the live cell data.

8. The method of claim 1, further comprises:
analyzing the second signal to obtain the proteome data.

9. The method of claim 1, wherein associating the live cell data and proteome data comprises at least one of: cohering, comparing, joining and subtracting the data.

10. The method of claim 1, further comprises:
generating a dataset comprising the chip data for a subset of the wells of the chip.

11. The method of claim 10, wherein the subset of the wells excludes one or more of: wells that are not occupied by at least one cell; and, wells that are occupied by more than one cell.

12. The method of claim 1, wherein the chip data is selected from the group consisting of: first signal; second signal; third signal; analyzed first signal; analyzed second signal; live cell data; proteome data; association between the live cell data and proteome data; and, any combination thereof.

13. The method of claim 1, further comprises:
determining the occupancy before performing live cell imaging.

14. The method of claim 1, wherein detecting the first and the second signal are performed at the same or different devices. The method of claim 1, wherein the cell suspension comprises up to 800,000 cells/m I.

16. The method according to claim 1, wherein the cell suspension comprises from to 500,000 cells/ml.

17. The method of claim 1, wherein the proteome cell content comprises: cell proteins; and, peptides.

18. The method of claim 2, wherein the DNA staining is effected using a fluorescent label.

19. The method of claim 18, wherein the fluorescent label of DNA is selected from: DAPI TOTO-1 iodide; Hoechst; 7-ADD; crystal violet; and, ethidium bromide.

20. The method of claim 18, wherein the respective DNA data for each well of the scWestern chip comprises a predetermined function of the fluorescence values from the respective well associated with the DNA staining.

21. The method of claim 20, wherein the predetermined function comprises a multiplication of:
an area under the curve (AUC) of the outcome of a convolution of a signal to noise ratio (SNR) signal and a predetermined peak shape, the SNR signal being of fluorescent counts in relation to a distance from the center of the respective well; and a width of the outcome of the convolution.

22. The method of claim 18, wherein the at least one predetermined threshold comprises a predetermined statistical attribute of the DNA data of the wells of the scWestern chip.

23. The method of claim 18, wherein the at least one predetermined threshold comprises a plurality of predetermined statistical attributes, each predetermined statistical attribute associated with a respective one of a plurality of sets of adjacent wells of the scWestern chip.

24. The method of claim 22, wherein the predetermined statistical attribute comprises a median value.

25. The method of claim 18, wherein the at least one predetermined threshold comprises the lowest value of the DNA data of the wells of the scWestern chip.

26. The method of claim 18 wherein the at least one predetermined threshold comprises the lowest value of the DNA data of each of a plurality of sets of adjacent wells of the scWestern chip.

27. The method of claim 18, wherein, for each of the wells of the scWestern chip, the method further comprises, responsive to an outcome of the comparison to the at least one predetermined threshold, determining whether the respective well is one of: occupied by one cell, occupied by more than one cell and not occupied.

28. The method of claim 27, further comprising, for each of the wells of the scWestern chip determined to be occupied by at least one cell, subtracting from the DNA data of the respective well a respective value associated with the DNA data of at least one well determined to not be occupied by at least one cell.

29. The method of claim 28, wherein the at least one well not occupied by at least one cell comprises the two of the wells closest to the respective well.

30. The method of claim 1, wherein live cell imaging comprises fluorescent live cell imaging.

31. The method of claim 1, wherein live cell imaging comprises bright field live cell imaging.

32. The method of claim 1, wherein determination of occupancy further comprises:
identifying wells containing no cells therein, one cell therein, and more than one cell therein.

33. The method of claim 30, further comprises:
marking each well containing no cells therein by a first marking;
marking each well containing one cell therein by a second marking; and
marking each well containing two or more cell therein by a third marking.
